(19) United States
(12) Patent Application Publication
(10) Pub. No.: US 2019/0240472 A1
(43) Pub. Date: Aug. 8, 2019

(54) ENHANCED PLEURAL THROMBOLYSIS

(71) Applicant: Ian MAKEY, San Antonio, TX (US)

(72) Inventor: Ian MAKEY, San Antonio, TX (US)

(73) Assignee: THE BOARD OF REGENTS OF THE UNIVERSITY OF TEXAS SYSTEM, Austin, TX (US)

(21) Appl. No.: 16/335,477

(22) PCT Filed: Sep. 21, 2017
(86) PCT No.: PCT/US2017/052764

§ 371 Date: Mar. 21, 2019

Related U.S. Application Data
(60) Provisional application No. 62/397,538, filed on Sep. 21, 2016.

(51) Int. Cl. A61M 37/00; A61N 7/00; A61M 25/00; A61B 17/22

(52) U.S. Cl. CPC A61M 37/0092; A61N 7/00; A61B 2017/22014; A61B 17/22004; A61M 25/0074; A61M 25/0082

(57) ABSTRACT

Embodiments of the devices described herein include a method and apparatus to deliver sound or vibration energy to enhance dissolution and drainage of loculated pleural effusions.

This Application claims priority to U.S. Provisional Patent Application Ser. No. 62/397,538 filed Sep. 21, 2016, which is incorporated herein by reference.

### BACKGROUND

Loculated pleural effusions remain a common and burdensome clinical entity, with the commonest causes being hemothorax and empyema. Many loculated pleural effusions can be treated with drainage alone whether via a percutaneous catheter or a surgical chest tube. However, there is a significant percentage of loculated effusions that fail to drain with this treatment.

Pleural infection, in the form of empyema, hospitalizes more than 24,000 patients each year in the United States and the incidence is increasing with the aging of the baby boomers and more antibiotic resistant bacteria. Approximately half of these patients will undergo surgical therapy to evacuate the pleural space. Hemothorax occurs most commonly after trauma. A rough estimate of the occurrence of hemothorax after trauma in the United States is 300,000 cases per year. Blood that remains in the chest after placement of a chest tube is called a retained hemothorax. Retained hemothorax occurs in up to 30% of patients with trauma related hemothorax for an annual incidence of 90,000 cases per year. Of these 40% undergo surgery. Therefore almost 50,000 operations are performed in the U.S. each year to treat unwanted effusions.

A less invasive solution to this problem has been sought for many years. One such approach is the use of solvents to dissolve the loculated fluid and permit drainage. These solvents are called fibrinolytics because they breakdown fibrin. Fibrin is what traps the fluid. Intrapleural fibrinolytic therapy has been attempted for infected pleural effusions since the late 1940's. Adoption was limited until better tolerated and more efficacious fibrinolytics were developed. This came in the form of tissue plasminogen activator (tPA) in 1982 and DNase in 1993.

Numerous studies have shown that fibrinolytics can be effective, however they are not the standard-of-care for loculated effusions. There are several reasons for this. The first reason is that the clinical benefit is inconsistent. Some pleural effusions respond quite well to fibrinolytic therapy whereas others do not respond at all. Complete failure of treatment occurs at least 10% of the time. Second, fibrinolytic therapy can be slow. The length of time needed to treat with fibrinolytics is usually 3 days or longer. If not successful then this causes a delay in definitive treatment. Finally, large doses of fibrinolytics can result in bleeding, which can exacerbate the existing problem. Fortunately technology exists to enhance fibrinolytics and speed the breakdown of thrombi. The device described herein is the first to describe the application of this technology to pleura effusions.

### SUMMARY

Embodiments of the devices described herein describe the application of sound or vibrational energy (vibration) to enhance the breakdown, dissolution, and drainage of unwanted effusions in the pleural cavity. There are two general groups of devices. The first group of devices (thrombolytic devices) apply ultrasound, vibration, or infrasound externally to the body or chest wall. They transmit sound or vibration through the chest wall into the pleura space or through the body or organ to a targeted tissue or body cavity. The simplest form is a hand-held thrombolytic device much like a diagnostic ultrasound device. In certain aspects the device is positioned and/or held in place by a person. Another form of this first type of external thrombolytic device is an ultrasound transmitter or other vibration source, which is positioned and/or held in place by a stationary arm or other positioning device. Additional embodiments of the first type of device include one or many transmitters or vibration sources positioned and/or held in place close to the body or chest wall in the form of an adhesive patch, wrap, harness, or vest. Another embodiment can use a diagnostic ultrasound to guide positioning while the same or a secondary device can deliver therapeutic sound or vibration to a target.

A second group of devices (internal devices) can be inserted into the body, or through chest wall and into the pleural space via a catheter, drainage catheter or as an embodiment of a drainage catheter (integrated with the catheter). One embodiment is an adjunct to chest tubes or drainage tubes. The transmitting element or vibration source would be shaped like a catheter so as to pass through a chest tube or drainage tube. Another embodiment is a transmitting element or vibration source combined with or embedded into/onto a drainage tube. This group of devices permits the ultrasound transmitter or vibration source to be in closer proximity to the target fluid than the first group of external devices. Associated benefits can include disbursement of the fibrinolytic agent into the target fluid more quickly when it originates from the same catheter as the ultrasound transmitter or vibration source. Also the sound or vibration energy may prevent clogging of the drainage catheter. The beauty of this design is that it would not require an additional invasive procedure. This is because one of the basic steps in treatment of unwanted pleural effusions is placement of a drainage catheter. The dissolved fluid can then drain either via dependent gravity drainage or suction via a standard chest tube canister (pleuravac) device.

The drainage tube has a proximal end configured to be outside the chest wall when deployed and a distal end configured to be inside a patient when in use. In certain aspects 1, 2, 3, 4, 5, 6, 7, 8, 9, 10 or more sound or vibration sources are positioned in the distal third of the chest tube or catheter, preferably in the distal 2, 3, 4, 5 cm of the chest tube or catheter. The sound source(s) or vibration source(s) (generally referred to as sources) can be aligned along the long axis of the chest tube or catheter or positioned circumferentially around tube wall or catheter. They can be positioned to form a spiral or other pattern of sources along the chest tube or catheter. In other embodiments a tunable source or a combination of sound (ultrasound/infrasound) and vibration sources can be used.

In certain aspects the source(s) may or may not be coupled to an external box or controller, e.g., via electrical wires. The sources not coupled to an external box or controller will have a self-contained power source and can be connected via wireless communication to a controller. The controller modulates the frequency and amplitude (i.e., power) of the sound or vibration energy form the source(s). The controller can activate one, two, or all of the transmitters or sources, as is found to be most therapeutic. In another embodiment, the ultrasound transducers or vibration sources may produce sound or vibration energy directionally, that has the effect of moving the drainage catheter within the body or pleural space. Thus, the controller can move the catheter by activating a sequence of directional transducers or sources.

The sound transmitters or vibration sources can be embedded or combined with the drainage tube and the lumen of the drainage tube or an associated delivery tube can be used as a delivery lumen for fibrinolysis reagents. The reagent can be delivered under pressure so as to provide additional physical force to loosen a target for removal. The reagent delivery lumen can end in 1, 2, 3, 4, 5, or more reagent delivery ports. Reagent delivery ports can end at the tip of the chest tube or associated delivery tube, in the distal end of the chest tube lumen, or to the external surface of the chest tube wall. In certain aspects, the reagent delivery lumen can be a separate lumen from the drainage lumen. 1, 2, 3, 4 or more delivery lumens can run along the long axis of the chest tube and can be positioned on the lumen side of the chest tube or on the external surface of the chest tube, or at the terminal edge of the chest tube.

Reagents used in conjunction with the devices described herein can include an antibiotic, antifungal, plasminogen activator, nuclease (e.g., DNase), protease, mucinase, urokinase, streptokinase, heparin, or other compounds or enzymes that dissolve or cleave a component of a pleural effusion, fibrinous septation, thrombus, or other pathologic fluid or formation that may be located in the intrapleural space or chest.

In one aspect, a method of treating can include positioning a chest tube or catheter as described herein at a treatment site and delivering one or more of reagent(s), sound energy, vibration energy, or sound and vibration energy. The method of treating can comprise passing an ultrasound, infrasound, and/or vibration capable chest tube or catheter through the patient's chest wall to the treatment site. The chest tube or catheter can include at least one distal fluid delivery port and at least one sound or vibration source.

As used herein, the terms “vibration” or “vibrational energy” refers to an oscillating, reciprocating, or other periodic motion of a rigid or elastic body, or a medium forced from a position or state of equilibrium. Vibrations can be at a frequency of between 0.01 Hz to 20 Hz to less than 20 kHz (including infrasound (frequencies below the human audible range) (<20 Hz)), whereas “ultrasonic energy,” “ultrasound,” and “ultrasonic” (frequencies above the human audible range) refers to energy sound at frequencies greater than 20 kHz to about 20 MHz. In certain aspects vibrations have a frequency of between 20 Hz and 20 kHz and infrasound have a frequency of 0.01 Hz or less to 20 Hz. Vibration, ultrasound, and/or infrasound can be emitted in a continuous or dis-continuous fashion (e.g., pulsed), depending on the parameters of a particular application. Additionally, the energy can be emitted in waveforms having various shapes, such as sinusoidal waves, triangle waves, square waves, or other wave forms. The vibration or sound sources span the frequency spectrum from infrasound (<20 Hz) to sound (20 Hz to 20 kHz) to ultrasound (>20 kHz). Some experiments shown that infrasound can be effective in enhancing the function of tPA to break down thrombus. In certain embodiments described herein, the time average acoustic power of the vibrational energy is between about 0.01, 0.1, 1 watts and 1.5, 2, 2.5, 3 watts per energy emitter.

As used herein, the term “source” refers to an device capable of producing various vibrational or sound energy. The source may be in the form of a motor with an eccentric weight. The sound emitter may be in the form of a membrane with magnetically induced motion. An ultrasonic transmitter or transducer converts electrical energy into ultrasonic energy, is an example of an ultrasound source. One example of a ultrasonic transducer capable of generating ultrasonic energy from electrical energy is a piezoelectric ceramic oscillator. Piezoelectric ceramics typically comprise a crystalline material, such as quartz, that changes shape when an electrical current is applied to the material. This change in shape, made oscillatory by an oscillating driving signal, creates ultrasonic sound waves.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1C. Illustration of two non-limiting examples of a thrombolytic chest tube; (1A) linear embodiment and (1C) pigtail embodiment; (1B) illustrates a cross section that is representative of both embodiments.

FIG. 2. Illustration of one embodiment of a vest supporting an therapeutic ultrasound device.

FIG. 3. Illustrates to two embodiments for circuits used to modulate or control ultrasound sources.

FIG. 4. Percent evacuation of blood in a test tube containing 30 cc of coagulated blood followed by 1 mg of tPA and sequential evaluation of drainage.

FIG. 5. Percent evacuation of blood in a test tube hemothorax model (n=8 in each group) using ultrasound with Definity contrast agent versus control. The test tube contained 30 cc of coagulated blood followed by 1 mg of tPA and a 1 hour dwell time. Error bars represent one standard deviation.

FIG. 6. Percent evacuation of blood in a test tube hemothorax model (n=1 in each group) using a oscillation flush method versus control. The test tube contained 30 cc of coagulated blood followed by 1 mg of tPA. The amount evacuated was measured sequentially over 4 hours.

FIG. 7. Percent evacuation of blood in a test tube hemothorax model (n=4 in each group) using motor vibration versus control. The test tube contained 30 cc of coagulated blood followed by 1 mg of tPA and a 1 hour dwell time. Error bars represent one standard deviation.

FIG. 8. Percent evacuation of blood in a pig hemothorax model (n=2 in each group) using three different methods of mechanical agitation versus a control chest tube. 500 cc of coagulated blood was instilled followed by 5 mg tPA and a 1 hour dwell time. Blue is the amount of blood evacuated and orange is the amount retained within the chest. Error bars represent one standard deviation.

FIG. 9. Percent evacuation of blood in a pig hemothorax model (n=2 in each group) with a vibration motor chest tube compared to a control chest tube after instillation of 500 cc of blood and a delay time of 15 minutes. Blue is the amount of blood evacuated and orange is the amount retained within the chest. Error bars represent one standard deviation.

FIG. 10. One embodiment of a thrombolytic catheter having a vibration source in proximal end of the catheter.

### DESCRIPTION

The basic science behind ultrasound-enhanced thrombolysis is proven. The technology is already being applied in various clinical settings such as deep vein thrombosis, arterial thrombosis (stroke, peripheral artery disease), pulmonary artery embolus, and sub-dural hematoma. Interestingly, loculated effusions in the pleural space may be the most suitable application. The pleural space is the largest potential space in the body and large, difficult to drain effusions accumulate there. Video-assisted thoracoscopic surgery (VATS) is an effective solution, but it has downsides. VATS requires a skilled surgeon, general anesthesia, and one-lung ventilation. Thrombolytic therapy is a promising solution, but consistency and effectiveness of treatment is questionable. Enhancing thrombolytic therapy with ultrasonic energy, with or without microbubbles, may be the extra step required to replace VATS in the treatment of loculated pleural effusions.

Ultrasonic energy focused upon a blood clot or loculated fluid causes it to break apart and dissolve. This process termed thrombolysis or fibrinolysis liquefies the clot or fluid and allows subsequent drainage through the drain. Depending on the frequency of the ultrasonic energy used, the ultrasound effect is carried through by means of mechanical action, heat or cavitation. The lower frequency acoustical waves, usually below 50 KHz, dissolve a blood clot or loculated fluid by cavitation and/or mechanical action. Frequencies above 500 KHz take affect more so by generating heat. The lower frequency waves have a wider area of affect whereas the higher frequency waves have a shorter area of affect.

The process by which thrombolysis or fibrinolysis is affected by use of ultrasound in conjunction with a thrombolytic agent can vary according to the frequency and power of the energy applied, as well as the type and dosage of the thrombolytic agents. The application of the ultrasound has been shown to cause reversible changes to the fibrin structure within the thrombus, increased fluid dispersion into the thrombus and facilitated enzyme kinetics. These mechanical effects beneficially enhance the rate of dissolution of thrombi.

It may be possible to reduce the typical dose of thrombolytic agent when ultrasonic energy is also applied. The ability to reduce the dosage of the thrombolytic agent when ultrasound is applied can potentially lead to fewer complications and an increased patient population eligible for treatment.

Ultrasound contrast agents are air or gas-filled microbubbles with a stabilizing shell constituted by a lipid shell monolayer. In the presence of ultrasound waves, these microbubbles oscillate. The oscillations of the microbubbles in turn induce increased movement of adjacent structures and increase the lytic rate of the reagent within a thrombus. The most common reagents applied in the pleural space are plasminogen activator (tPA) and DNase. The mechanical agitation improves drug penetration and accessibility of fibrin structures to clot-dissolving enzymes. tPA plus ultrasound waves plus microbubbles have been shown to have a very high thrombolytic rate.

Devices for assisting the treatment of the conditions described herein include external ultrasound device, ultrasound capable catheters, ultrasound capable chest tubes, or combinations thereof.

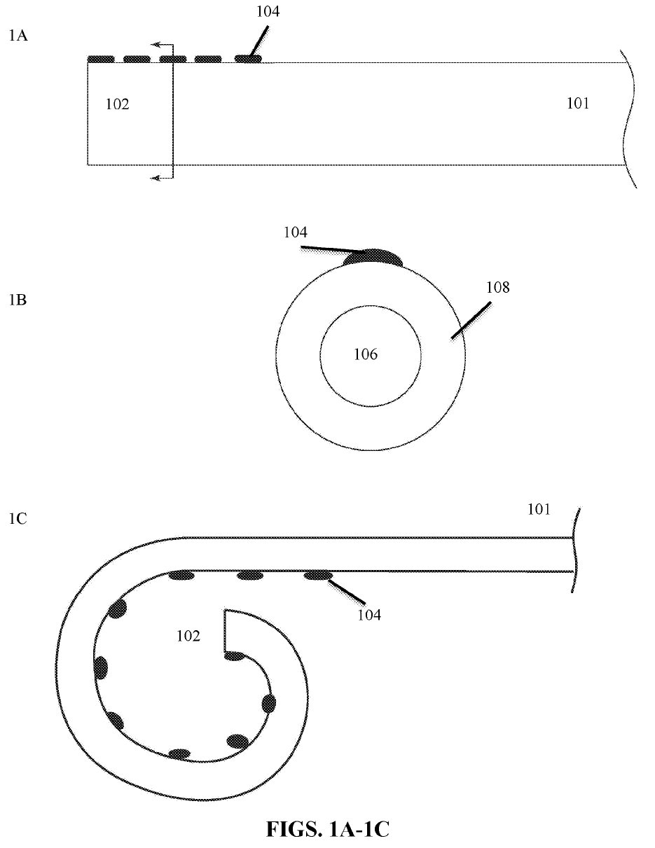

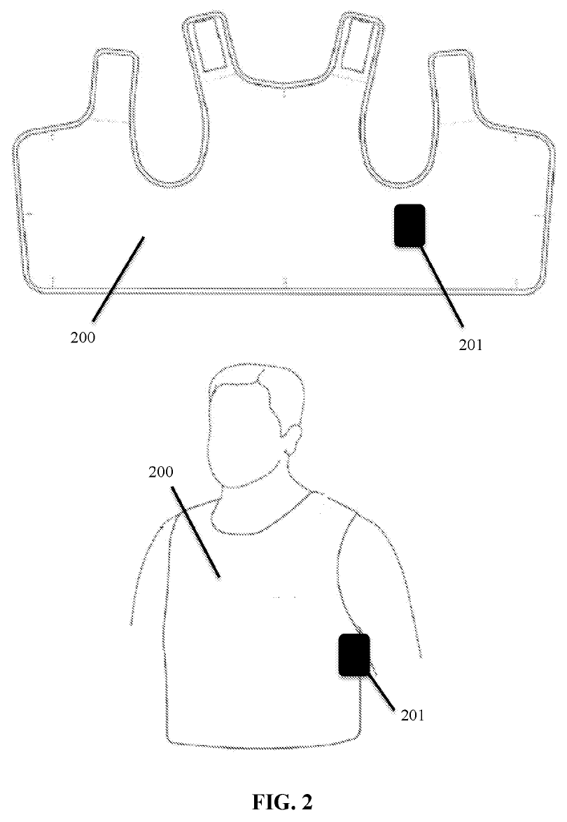

FIG. 1 illustrates two examples that demonstrate some aspects of the invention. Illustrated are two embodiments of a chest tube configured to provide acoustic energy. A first embodiment is a linear chest tube (FIG. 1A) and a second embodiment is a pigtail chest tube (FIG. 1C). Each of chest tubes have a tube wall 108 that forms an interior lumen 106 (FIG. 1B). The chest tube can be configured with an acoustic source 104. The chest tube can comprise 1, 2, 3, 4, 5, 6, 7, 8, 9, 10, or more acoustic sources 104. The acoustic sources can be aligned along the long axis of the chest tube or positioned circumferentially around the tube wall 108. In certain aspects the acoustic source can be positioned along the long axis and circumferentially, which can form a spiral or other pattern of acoustic sources along the chest tube. In certain aspect an acoustic source can be position at the distal end 102 of a chest tube. The acoustic sources can be configured to be activated individually or as a group or subgroup. In certain aspects activating certain transducers and not others can control the directionality of the acoustic energy.

In another embodiments a reagent lumen that connects a distal 102 reagent port to a proximal 101 access port is provided. The reagent lumen is configured to allow a reagent to be introduced at or with 1, 2, 3, 4, or 5 cm of the proximal end 102. In certain aspects the reagent can be provided as a drip or under pressure. The reagent can be provided as a fluid or liquid under about 5, 10, 20, or more psi of pressure. If the reagent is provided under pressure it can be used as a physical force to aid in breaking and removing a target. The ultrasound transducers can be embedded in the chest tube wall or lumen. Electrical wires connect the transducers to an external electrical source.

FIG. 2 illustrates one embodiment of a device for positioning an ultrasound source externally. As shown in FIG. 2 the device can be vest 200 that has at least one ultrasound source 201 that can be coupled to a controller. A vest can be designed to fit around the shoulders of a patient and be worn around the chest of a patient. Other embodiments can be in the form of a belt that can be wrapped around and fastened to the chest and below the armpits. Such devices can have 1, 2, 3, 4, 5, 6, 7, 8, 9, 10 or more ultrasounds sources that can be controlled as a group or individually.

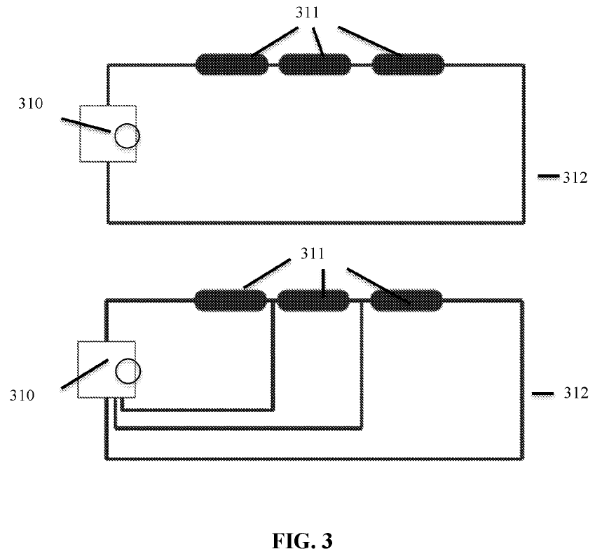

FIG. 3 diagrams certain circuitry 312 for control of the ultrasound sources 311. Controller 310 can be coupled to one or more ultrasound sources in parallel of series enabling the control of ultrasound sources 311 as a group or individually.

### EXAMPLES

The following examples as well as the figures are included to demonstrate preferred embodiments of the invention. It should be appreciated by those of skill in the art that the techniques disclosed in the examples or figures represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

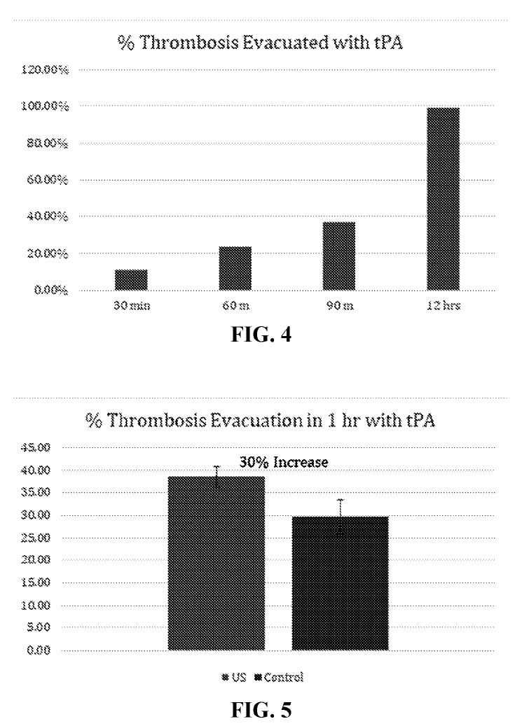

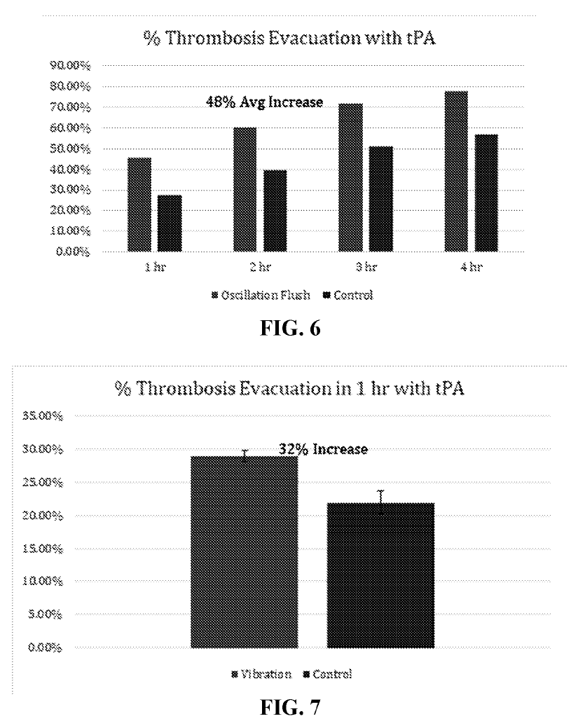

The inventor performed multiple benchtop experiments using clotted blood in a test tube model. Using these results, tissue plasminogen activator (tPA) was found to be a potent thrombolysis agent very dependent on time (FIG. 4). In addition to time, mechanical agitation was found to also be a potent factor in thrombolysis. Ultrasound, oscillating flushing, and vibration motor all enhanced tPA thrombolysis (FIGS. 5, 6, and 7).

In-Vivo Experiments:

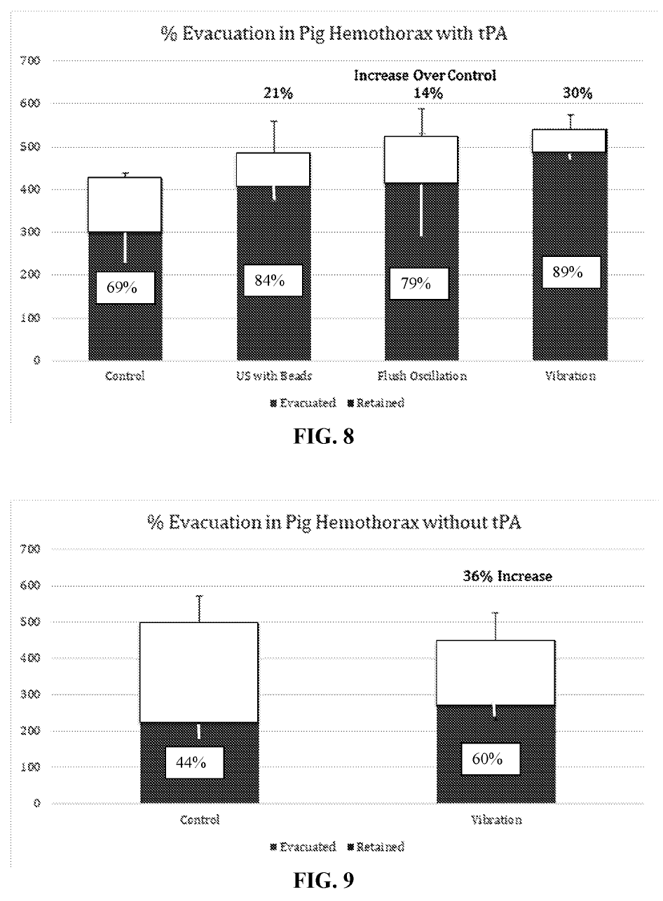

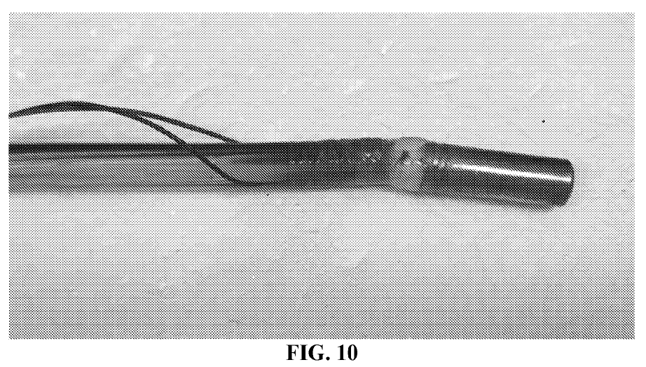

Based on results from the benchtop experiments, the inventor performed a pilot in-vivo study to determine which agitation method worked best. Prototypes were built for each method. Each method was its own experimental group. There was 1 control group. Each group was composed of two pigs. As with the benchtop experiments, the experimental groups evacuated blood more effectively than the control group (FIG. 8), mechanical agitation in combination with tPA drained a hemothorax more effectively than tPA alone.

From the pilot study, it was determined that of the three experimental groups, the vibration motor chest tube was the most effective. Therefore, the inventor devised an experiment to see if the vibration motor chest tube would drain blood better than a conventional chest tube without the presence of tPA. The vibration motor chest tube drained 36% more blood than the control chest tube (FIG. 9).

Database Analysis:

Using tPA to drain unwanted collections in the pleural space, such as hemothorax or empyema, may eventually replace surgical therapy. Surgical therapy currently consists of video assisted thoracoscopic surgery (VATS) to evacuate the pleural space. There have been case series describing the outcomes of VATS procedures and intra-pleural instillation of WA to treat these conditions, but no database studies. Therefore, generalized outcomes of VATS procedures and intra-pleural instillation of tPA for empyema and hemothorax is unknown. Using a database approach, the inventor sought to define the risks and outcomes of the standard therapy compared to a new therapy.

Results and Discussion:

The combination of in-vitro and in-vivo experiments confirms that mechanical agitation does increase the effectiveness of tPA in a clotted hemothorax model. Based on the in-vivo experiments, the motor vibration prototype worked the best with 30% more blood evacuated than the control followed by ultrasound (21%) and oscillation flush (14%) (FIG. 8). It was suspected that the improvement derives from mechanical disruption of the blood thrombus which increases the surface area available for tPA to join with fibrin. Once joined with fibrin, tPA's catalytic activity increases (Hoylaerts et al., J Biol Chem. 1982, 257:2912-19; Ranby, Biochim Biophys Acta. 1982, 704:461-69). This process was clearly evident when tPA and a blood clot were “stirred” with manual intervention. With manual intervention, the thrombolysis effect was consistently doubled or tripled (data not shown). The key problem is transmitting this “stirring” effect to a blood clot within a human chest without injuring the lung or other organs. All the methods developed are transmissible through a chest tube. Placement of a chest tube is standard practice in the treatment of unwanted collections in the chest. Having performed the in-vivo pig experiments, it was found that the treatments are safe and pose no risk of harm beyond the bleeding risk associated with tPA. Moreover, it was suspected that exposing more target blood clot to tPA binding would lower the amount of tPA available for iatrogenic fibrinolysis and bleeding.

A vibration motor attached to a chest tube, improved blood drainage by 36% independent of the presence of tPA (FIG. 9). The inventor thought that the vibration would lengthen the time to clot formation in fresh blood, however this was not the case (data not shown). Therefore, the exact mechanism of improve drainage is not known at this time. The data for tPA independent drainage is applicable to almost all drainage tubes.

1. A thrombolytic device comprising (a) sound or vibration source, and (b) an external positioning apparatus.

2. The device of claim 1, wherein the external positioning apparatus is a vest or belt, or an adjustable arm.

3. (canceled)

4. A method for treating pleural effusions comprising exposing a target effusion to ultrasound energy.

5. The method of claim 4, further comprising providing sound or vibration energy from an external source positioned against the chest.

6. The method of claim 4, further comprising providing sound or vibration energy from an internal source positioned in the chest.

7. The method of claim 6, wherein the sound or vibration source is an sound or vibration capable catheter or chest tube.

8. The method of claim 4, further comprising administering a thrombolytic reagent.

9. The method of claim 8, wherein the thrombolytic agent comprises plasminogen, DNAase, micro-bubbles, heparin, saline, or distilled water.

10. A thrombolytic catheter for disrupting pleural effusions comprising:
an elongate flexible catheter body having a proximal portion, a distal portion; and
an sound or vibration source positioned in the distal 5 cm of the catheter body;
wherein the sound or vibration source is capable of coupling with a controller.

11. The catheter of claim 10, wherein the outer diameter is about 7 french or less.

12. The catheter of claim 10, further comprising a reagent delivery lumen.

13. A thrombolytic chest tube comprising:
a wall forming an elongated lumen having a proximal and distal portion, the proximal portion ending in a proximal opening and the distal portion ending in a distal opening; and
the distal portion comprising at least one sound or vibrational source configured to provide energy in the form of sound or vibration to a target.

14. The tube of claim 13, further comprising a reagent delivery port at the distal end that is in fluid communication with an access port in the proximal end of the chest tube via a reagent delivery path, the reagent delivery path is configured to introduce a reagent from the proximal end to the distal end of the chest tube.

15. The tube of claim 13, wherein the sound or vibration energy propagates at an angle of 0 to 90 degrees with respect to the long axis of the tube.

16. The tube of claim 13, wherein the tube is 10, 20, 30, 40, 50 cm in length.

17. The tube of claim 13, wherein the tube has a lumen of 8 to 45 French.

18. The tube of claim 13, wherein the tube is a polyvinylchloride or silicon tube.

19. (canceled)

20. The tube of claim 13, wherein the sound or vibration source is connected to a controller by wiring.

21. (canceled)

22. The tube of claim 20, wherein the controller is programmed to activate the sound or vibration source in continuous or pulsed wave pattern.
